(19) United States
(12) Patent Application Publication
(10) Pub. No.: US 2017/0335216 A1
(43) Pub. Date: Nov. 23, 2017

(54) BASE OIL HAVING HIGH VISCOSITY INDEX FROM ALKYLATION OF DIMER KETONE-DERIVED OLEFIN

(71) Applicant: Chevron U.S.A. Inc., San Ramon, CA (US)

(72) Inventor: Sven Ivar Hommeltoft, Pleasant Hill, CA (US)

(21) Appl. No.: 15/158,750

(22) Filed: May 19, 2016

(51) Int. Cl. C10M 105/04; C07C 29/145; C07C 1/24; C07C 2/62

(52) U.S. Cl. CPC C10M 105/04; C07C 2/62; C07C 29/145; C07C 1/24; C07C 2531/02; C10M 2203/022; C07C 2521/04

(57) ABSTRACT

A process to make an alkylate base oil having a viscosity index greater than or equal to 90, comprising:
a. converting an at least one dimeric ketone to an at least one alcohol;
b. dehydrating the at least one alcohol to make one or more corresponding olefins; and
c. alkylating at least one isoalkane with the one or more corresponding olefins to form the alkylate base oil.

This application is related to two co-filed applications titled “FARNESANE ALKYLATION”, and “HIGH VISCOSITY INDEX LUBRICANTS BY ISOALKANE ALKYLATION”, herein incorporated in their entireties.

### TECHNICAL FIELD

This application is directed to a process for making high quality alkylate base oil from a dimeric ketone by converting the dimeric ketone to an alcohol, dehydrating the alcohol to make olefins, and alkylating an isoalkane with the olefins.

### BACKGROUND

Improved processes for making high viscosity index alkylate base oils from feeds comprising one or more dimeric ketones are needed.

### SUMMARY

This application provides a process to make an alkylate base oil having a viscosity index greater than or equal to 90, comprising:

a. converting an at least one dimeric ketone to an at least one alcohol;

b. dehydrating the at least one alcohol to make one or more corresponding olefins; and

c. alkylating at least one isoalkane with the one or more corresponding olefins to form the alkylate base oil.

The present invention may suitably comprise, consist of, or consist essentially of, the elements in the claims, as described herein.

### Glossary

“Base oil” refers to a hydrocarbon fluid to which other oils or substances are added to produce a lubricant.

“Lubricant” refers to substances (usually a fluid under operating conditions) introduced between two moving surfaces so as to reduce the friction and wear between them.

“Viscosity index” (VI) is an empirical, unit-less number that represents the temperature dependency of a lubricant, as determined by ASTM D2270-10(E2011). A higher VI indicates a smaller decrease in kinematic viscosity with increasing temperature of the lubricant.

“Predominantly” refers to greater than 50 wt % in the context of this disclosure.

“American Petroleum Institute (API) Base Oil Categories” are classifications of base oils that meet the different criteria shown in Table 1:

TABLE 1

| API Group | Sulfur, wt % | Saturates, wt % | Viscosity Index |
|---|---|---|---|
| I | >0.03 and/or | <90 | 80-119 |
| II | ≦0.03 and | ≧90 | 80-119 |
| III | ≦0.03 and | ≧90 | ≧120 |

| | |
|---|---|
| IV | All Polyalphaolefins (PAOs) |
| V | All base oils not included in Groups I-IV(naphthenics, |
| | non-PAO synthetics) |

“Group II+” is an unofficial, industry-established ‘category’ that is a subset of API Group II base oils that have a VI greater than 110, usually 112 to 119.

“Catalytic dewaxing”, or “hydroisomerization”, refers to a process in which normal paraffins are isomerized to their more branched counterparts in the presence of hydrogen and over a catalyst.

“Kinematic viscosity” refers to the ratio of the dynamic viscosity to the density of an oil at the same temperature and pressure, as determined by American Society for Testing and Materials (ASTM) D445-15.

“LHSV” means liquid hourly space velocity.

“Periodic Table” refers to the version of the IUPAC (International Union of Pure and Applied Chemists) Periodic Table of the Elements dated Jun. 22, 2007, and the numbering scheme for the Periodic Table Groups is as described in Chemical And Engineering News, 63(5), 27 (1985).

“Dimeric ketone” refers to a class of organic compounds containing a carbonyl group, CO, attached to two alkyl or alkenyl groups (R1 and R2), such as R1COR2. The two alkyl or alkenyl groups are either identical or similar (not necessarily identical) subunits or monomers.

“Carboxylic acids” are organic acids characterized by the presence of one or more carboxyl groups in their molecules. A carboxyl group consists of a carbon atom attached to an oxygen atom with a double covalent bond and to a hydroxyl group by a single covalent bond. The chemical formula of the carboxyl group may be written as —C(═O)OH, —COOH, or —CO₂H. “Transition metal” refers to any element in any of the series of elements with atomic numbers 21-29, 39-47, 57-79, and 89-107, that in a given inner orbital has less than a full quota of electrons.

“Corresponding” in the context of this disclosure means having the same carbon number as the hydrocarbon feed from which the hydrocarbon product, e.g., olefin or alcohol, is converted from.

“Acidic ionic liquid” refers to materials consisting entirely of ions, that can donate a proton or accept an electron pair in reactions, and that are liquid below 100° C.

“Naphtha range hydrocarbons” include the following products:

| | Typical Cut Points, ° F. (° C. ) |
|---|---|
| Products | for North American Market |
| Light Naphtha | C₅-180 (C₅-82) |
| Heavy Naphtha | 180-300 (82-149) |

“Cut point” refers to the temperature on a True Boiling Point (TBP) curve at which a predetermined degree of separation is reached.

“TBP” refers to the boiling point of a hydrocarbonaceous feed or product, as determined by ASTM D2887-13.

### DETAILED DESCRIPTION

The alkylate base oil made by this process has a high viscosity index, which makes it valuable for blending into a wide variety of finished lubricants. In one embodiment, the alkylate base oil has a viscosity index greater than or equal to 90, such as from 90 to 200. In another embodiment, the alkylate base oil has a viscosity index greater than or equal to 120. The alkylate base oil also has a kinematic viscosity at 100° C. that makes it useful for blending into finished lubricants. In one embodiment, the kinematic viscosity at 100° C. is greater than 1.8 mm²/s, such as from 2.0 to 25 mm²/s, or from 3.0 to 20 mm²/s.

In one embodiment, the alkylate base oil is an API Group II+ or an API Group III base oil.

In one embodiment, the process additionally comprises blending the alkylate base oil with at least one additive to make a finished lubricant. A wide variety of high quality finished lubricants can be made by blending the alkylate base oil with at least one additive selected from the group consisting of antioxidants, detergents, anti-wear agents, metal deactivators, corrosion inhibitors, rust inhibitors, friction modifiers, anti-foaming agents, viscosity index improvers, demulsifying agents, emulsifying agents, tackifiers, complexing agents, extreme pressure additives, pour point depressants, and combinations thereof; wherein selection of the at least one additive is directed largely by the end-use of the finished lubricant being made, wherein said finished lubricant can be of a type selected from the group consisting of engine oils, greases, heavy duty motor oils, passenger car motor oils, transmission and torque fluids, natural gas engine oils, marine lubricants, railroad lubricants, aviation lubricants, food processing lubricants, paper and forest products, metalworking fluids, gear lubricants, compressor lubricants, turbine oils, hydraulic oils, heat transfer oils, barrier fluids, and other industrial products. In one embodiment, the alkylate base oil can be blended with at least one additive to make a multi-grade engine oil.

In one embodiment, the alkylate base oil has a higher molecular weight, a lower pour point, and a lower cloud point than the at least one dimeric ketone. Pour point can be determined by ASTM D5950-14. Cloud point can be determined by ASTM-2500-16, by ASTM D7683-11, or by other automatic test methods for cloud point of petroleum products that give results similar to those in ASTM D2500-16, when they are bias corrected (as needed) according to their associated ASTM test method.

Bromine Index

In one embodiment, the alkylate base oil has a bromine index less than 1000, such as from 100 to 999, and a kinematic viscosity at 100° C. greater than 3 mm²/s. Bromine index can be determined by proton Nuclear Magnetic Resonance (NMR). Proton NMR is generally taught in https://en.wikipedia.org/wiki/Proton_nuclear_magnetic_resonance.

The following assumptions are made for the Bromine index determinations in test samples of alkylate base oil:

1) Residual olefins in the test sample are represented by the formula: R1R2C═CHR3, so that one vinylic hydrogen represents an olefin group.

2) The average carbon in the test sample caries two protons and thus may be represented by an average molecular wt of 14.0268 g/mole

3) All proton resonances in the range 0.5-0.95 represent methyl groups (3 protons per carbon)

4) All proton resonances in the range 0.95-1.40 ppm represent CH₂ groups (2 protons per carbon)

5) All proton resonances in the range 1.4-2.1 ppm represent CH groups (1 proton per carbon)

6) All proton resonances in the range 4-6 ppm represent RR′C═CHR″ groups (0.5 proton per carbon or one per double bond).

7) One double bond reacts with one equivalent of bromine, i.e., one mole of olefin reacts with one mole of dibromine (Br₂, MW=159.8 g/mole)

Integrals in the acquired proton NMR spectrum are represented by I(“group”), e.g., the integral of a methyl group is I(CH3) and the integral of an olefin group is I(RR′C═CHR″).

Bromine number is defined as the amount of bromine (in g Br₂) needed to titrate all the olefins in 100 g of the test sample. Bromine index=1000*bromine number.

The bromine index is calculated from the proton NMR integrals with the following formula: Bromine index=1000*100*(159.8/14.0268)*I(RR′C+CHR″)/{0.3333*I(CH3)+0.5*I(CH2)+I(CH)+2*I(RR′C═CHR″)}.

The absence of any proton resonances in the NMR spectrum is interpreted as a bromine index<100, based on the sensitivity of the proton NMR spectrometer that is used.

In one embodiment, the alkylate base oil has a kinematic viscosity at 100° C. between 4.0 and 6.0 mm²/s, a viscosity index from 175 to 195, and a pour point less than −20° C.

In one embodiment, the alkylating introduces branching into the alkylate base oil at a central position such that the alkylate base oil has a pour point less than −15° C. The positioning of the branching in the alkylate base oil can be determined by analyzing a sample of the alkylate base oil using ¹³C NMR (nuclear magnetic resonance).

In one embodiment, R1 and R2 in the dimeric ketone (R1COR2) are independently selected from the group consisting of C5-C21 linear or branched alkyl and C5-C21 linear or branched alkenyl.

In one embodiment, the dimeric ketone has a melting point greater than 65° C. Melting point can be determined by ASTM D5440-93 (R2009).

In one embodiment, the at least one dimeric ketone is prepared by ketonization of one or more carboxylic acids. Carboxylic acids are widespread in nature. Lower straight-chain aliphatic carboxylic acids, as well as those of even carbon number (up to C18), are commercially available. In one embodiment, the one or more carboxylic acids comprise long chain carboxylic acids that are obtained by the hydrolysis of triglycerides obtained from plant, algae, zooplankton, or animal oils.

In one embodiment, the at least one dimeric ketone is derived from a biological source, such as those containing fatty carboxylic acids. Examples of biological sources of fatty carboxylic acids include (but are not limited to) lard, chicken fat, beef tallow, coconut oil, cocoa butter, palm oil, palm kernel oil, cottonseed oil, wheat germ oil, jojoba oil, soybean oil, olive oil, corn oil, sunflower oil, safflower oil, hemp oil, canola oil, and mixtures thereof.

In one embodiment, the at least one dimeric ketone is prepared by ketonization of one or more carboxylic acids. In one embodiment, the at least one dimeric ketone can be made by: contacting at least one fatty acid with a ketonization catalyst in a ketonization zone under ketonization conditions to provide a long chain dimeric ketone according to the following Scheme 1:

R₁COOH+R₂COOH→R₁C(O)R₂+CO₂+H₂O

wherein R1 and R2 are independently selected from the group consisting of C5-C21 linear or branched alkyl and C5-C21 linear or branched alkenyl.

Converting

The at least one dimeric ketone is converted to an at least one alcohol. In one embodiment, the converting of the at least one dimeric ketone to an at least one alcohol is done by contacting the at least one dimeric ketone with a chemical reducing agent. Chemical reducing agents that can be used include organic reducing agents and inorganic reducing agents. Examples of organic reducing agents include: isopropanol and other secondary alcohols, and sugars. Examples of inorganic reducing agents include sodium borohydride, hydrazine, lithium, aluminum hydride, hydroxylamine, and sodium hypophosphite.

In one embodiment, the converting is done by contacting the at least one dimeric ketone with a hydrogen and a solid hydrogenation catalyst. Some solid hydrogenation catalysts include those comprising a transition metal on a support.

Solid hydrogenation catalysts include, but are not limited to, metal catalyst systems having active components comprised of Fe, Ni, Co, Cu, Cr, Mo, Sn, or W; noble metal catalyst systems having active components comprised of Pt, Pd, Rh, Ru, Re, or Ir; and combinations of these solid hydrogenation catalysts. Suitable supports for the solid hydrogenation catalyst include, but are not limited to, alumina, silica, silica-alumina, and carbon.

The solid hydrogenation catalyst can comprise from about 0.1 to about 98 wt % of the active components. Hydrogenation activity can be controlled by the exposed metal surface area of such catalysts. In one embodiment, the desired metal weight loading of the solid hydrogenation catalyst can be governed by the activity desired in the converting step. In one embodiment, the metal weight loading in the metal-loaded solid hydrogenation catalyst may be from about 0.1 to about 10.0 wt %. In one embodiment, the solid hydrogenation catalyst used for the converting provides a selectivity to make an at least one corresponding alcohol of 80 wt % or more, such as 80 to 99 wt %.

In one embodiment, the solid hydrogenation catalyst is a carbon supported metal hydrogenation catalyst. In one embodiment the carbon supported metal hydrogenation catalyst comprises one or more metals from the group consisting of Ru, Pt, and Cu.

In one embodiment, the converting is done at high selectivity, such that greater than 50 wt % of the dimeric ketone is converted to at least one alcohol. In one embodiment, a carbon supported metal hydrogenation catalyst is used for the contacting of the at least one dimeric ketone. For example, a carbon supported metal hydrogenation catalyst comprising from 0.1 to 10 wt % hydrogenation metal can be used to obtain a conversion of the at least one dimeric ketone to the at least one alcohol of greater than 80 wt %.

One way to do the converting of the at least one dimeric ketone to an at least one alcohol is by contacting the at least one dimeric ketone with a selective ketone hydrogenation catalyst in a ketone hydrogenation zone in the presence of hydrogen gas under selective ketone hydrogenation conditions to provide a long chain secondary alcohol according to the following Scheme 2:

R₁C(O)R₂+H₂→R₁′CH(OH)R₂′

In one embodiment,

R1 and R2 are the same or different,

when R1 is alkyl, R1′=R1,

when R2 is alkyl, R2′=R2,

when R1 is alkenyl, R1′ is alkyl or alkenyl,

when R2 is alkenyl, R2′ is alkyl or alkenyl, and

R1 and R1′ have an equal number of carbon atoms, and R2 and R2′ have an equal number of carbon atoms.

In one embodiment the selective ketone hydrogenation conditions include one or more of the following: a temperature from 0° C. to 300° C., a hydrogen pressure from 300 to 20000 kPa, a LHSV from 0.1 to 5, and a residence time from 2 minutes to 48 hours.

Dehydrating

The process to make the alkylate base oil additionally includes dehydrating the at least one alcohol to make one or more corresponding olefins. A dehydration unit can be used to perform the dehydrating, and the dehydration unit can comprise a dehydration catalyst. The dehydrating can be done by contacting the at least one alcohol with the dehydration catalyst in the dehydration unit under dehydration conditions.

In one embodiment, the dehydrating is done with a dehydration catalyst comprising alumina. In one embodiment, the dehydrating is done with a dehydration catalyst comprising at least 90 wt % alumina. In an embodiment, the dehydration catalyst may be selected from the group consisting of alumina and amorphous silica-alumina. In a sub-embodiment, the dehydration catalyst may comprise alumina doped with an element selected from the group consisting of phosphorus, boron, fluorine, zirconium, titanium, gallium, magnesium, and combinations thereof. In another sub-embodiment, the dehydration catalyst may comprise amorphous silica-alumina doped with an element selected from the group consisting of phosphorus, boron, fluorine, zirconium, titanium, gallium, magnesium and combinations thereof.

In one embodiment, after the dehydrating of the at least one alcohol to make one or more corresponding olefins, an olefin-enriched hydrocarbon stream comprising the one or more corresponding olefins has 0-1 wt % oxygen.

In one embodiment, the degree of acidity of the dehydration catalyst may be selected, e.g., by the judicious doping of alumina or amorphous silica-alumina, to determine the conversion of the at least one alcohol to the corresponding olefin and/or also to control the proportion of alpha-olefins to total olefins in the olefin enriched hydrocarbon stream. The olefin composition of the olefin enriched hydrocarbon stream may in turn determine the composition of the alkylate base oil during the subsequent alkylating step.

In one embodiment, the one or more corresponding olefins have a carbon number from C11 to C43, such as from C19 to C35.

In one embodiment, the dehydration conditions include one or more of the following: a temperature from 176.7° C. (350° F.) to 482.2° C. (900° F.), a pressure from 0.06895 kPa (0.01 psia) to 689.5 kPa (100 psia), and a LHSV from 0.1 hr⁻¹ to 20 hr⁻¹. In one embodiment the dehydration is conducted in the presence an inert diluent (such as for instance a light hydrocarbon, nitrogen, or CO₂) at a molar ratio of the inert diluent to the at least one alcohol in the range of 0.5:1 to 20:1, and at a LHSV of 0.1 hr⁻¹ to 20 hr⁻¹. In one embodiment, the dehydration can be done under continuous operating conditions.

Hydroisomerization

In one embodiment, the process to make the alkylate base oil additionally includes hydroisomerizing the alkylate base oil with a hydroisomerization catalyst to lower a pour point of the alkylate base oil.

Hydroisomerization Catalysts

In a sub-embodiment, the hydroisomerization catalysts used in carrying out the hydroisomerization step include at least one dewaxing catalyst support, one or more noble metals, one or more molecular sieves, and optionally one or more promoters.

In a sub-embodiment, the dewaxing catalyst support is selected from the group consisting of alumina, silica, zirconia, titanium oxide, magnesium oxide, thorium oxide, beryllium oxide, alumina-silica, alumina-titanium oxide, alumina-magnesium oxide, silica-magnesium oxide, silica-zirconia, silica-thorium oxide, silica-beryllium oxide, silica-titanium oxide, titanium oxide-zirconia, silica-alumina-zirconia, silica-alumina-thorium oxide, silica-alumina-titanium oxide or silica-alumina-magnesium oxide, preferably alumina, silica-alumina, and combinations thereof.

In a sub-embodiment, the dewaxing catalyst support is an amorphous silica-alumina material in which the mean mesopore diameter is between 70 Å and 130 Å.

In another sub-embodiment, the dewaxing catalyst support is an amorphous silica-alumina material containing SiO2 in an amount of 10 to 70 wt. % of the bulk dry weight of the dewaxing catalyst support as determined by ICP (inductively coupled plasma) elemental analysis, a BET surface area of between 450 and 550 m2/g and a total pore volume of between 0.75 and 1.05 mL/g.

In another sub-embodiment, the dewaxing catalyst support is an amorphous silica-alumina material containing SiO2 in an amount of 10 to 70 wt % of the bulk dry weight of the dewaxing catalyst support as determined by ICP elemental analysis, and having a Brunauer, Emmett and Teller (BET) surface area of between 450 m²/g and 550 m²/g, a total pore volume of between 0.75 and 1.05 mL/g, and a mean mesopore diameter between 70 Å and 130 Å.

In a sub-embodiment, the amount of dewaxing catalyst support in the hydroisomerization catalyst is from 5 wt % to 80 wt % based on the bulk dry weight of the hydroisomerization catalyst.

In a sub-embodiment, the hydroisomerization catalyst may optionally contain one or more molecular sieves selected from the group consisting of SSZ-32, small crystal SSZ-32 (SSZ-32x), SSZ-91, ZSM-23, ZSM-48, EU-2, MCM-22, ZSM-5, ZSM-12, ZSM-22, ZSM-35 and MCM-68-type molecular sieves, and mixtures thereof. SSZ-91 is described in U.S. patent application Ser. No. 14/837,071, filed on Aug. 27, 2015. In one embodiment, the hydroisomerization catalyst may optionally contain a non-zeolitic molecular sieve. Examples of non-zeolitic molecular sieves which can be used include silicoaluminophosphates (SAPOs), ferroaluminophosphate, titanium aluminophosphate and various ElAPO molecular sieves. In the ElAPO molecular sieves the additional elements Li, Be, B, Ga, Ge, As, or Ti have been incorporated into the aluminophosphate framework.

In a sub-embodiment, the amount of molecular sieve material in the hydroisomerization catalyst can be from 0 wt % to 80 wt % based on the bulk dry weight of the hydroisomerization catalyst. In a sub-embodiment, the amount of molecular sieve material in the hydroisomerization catalyst is from 0.5 wt % to 40% wt %. In a sub-embodiment, the amount of the molecular sieve material in the hydroisomerization catalyst is from 35 wt % to 75 wt %. In a sub-embodiment, the amount of the molecular sieve material in the hydroisomerization catalyst is from 45 wt % to 75 wt %.

In a sub-embodiment, the hydroisomerization catalyst contains one or more noble metals selected from the group consisting of elements from Group 10 of the Periodic Table, and mixtures thereof. In a sub-embodiment, each noble metal is selected from the group consisting of platinum (Pt), palladium (Pd), and mixtures thereof.

In one embodiment, the process can additionally comprise passing the one or more corresponding olefins over an olefin isomerization catalyst to shift a double bond position to another internal position, without structurally introducing branching in the one or more corresponding olefins prior to the alkylating. The double bond shift can improve the possibility to form the alkylate base oil with a more diverse hydrocarbon composition in the subsequent alkylation step. A more diverse hydrocarbon composition in the alkylate base oil can favor better cold flow properties (e.g., lower pour point, lower cold-cranking simulator apparent viscosity, or lower pumping viscosity by mini-rotary viscometer).

Olefin isomerization catalysts that can be used to shift the double bond include those described previously for hydroisomerization, such as noble metal catalysts such as palladium or palladium (or mixtures thereof) on an alumina support, and other moderately acidic catalysts. Examples of suitable olefin isomerization catalysts are described in U.S. Pat. No. 8,198,494B2. The olefin isomerization catalyst may comprise a silicoaluminophosphate molecular sieve as the support. The olefin isomerization catalyst may comprise a zeolitic molecular sieve as the support. In certain sub-embodiments, the silicoaluminophosphate molecular sieve in the olefin isomerization catalyst can be SM-3, SAPO-11, SAPO-31 and/or SAPO-41. In certain sub-embodiments, the zeolitic molecular sieve in the olefin isomerization catalyst can be ZSM-5, ZSM-22, ZSM-23 and/or ZSM-35. In one embodiment, the olefin isomerization catalyst may comprise an intermediate size molecular sieve as the support, such as a molecular sieve with a pore size between 5.3 Å and 6.5 Å, when the porous inorganic oxide is in the calcined form.

Alkylating an Isoalkane

In one embodiment, the at least one isoalkane that is alkylated with the one or more corresponding (i.e., ketone derived) olefins to form the alkylate base oil has greater than or equal to four carbon atoms. For example, the isoalkane can have from four to 36 carbon atoms, or from five to 36 carbon atoms.

In one embodiment, the at least one isoalkane comprises farnesane, one of the isomers of which has the following chemical structure:

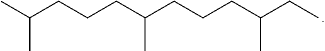

In one embodiment, the at least one isoalkane comprises a farnesane or an isopentane. In another embodiment, the at least one isoalkane comprises a mixture of naphtha range hydrocarbons.

In one embodiment, the at least one isoalkane comprises a hydrogenated dimer, trimer or oligomer of a light olefin such as propylene, butene or pentene. Examples of such isoalkanes include for instance 2-methylpentane (hydrogenated propylene dimer), 2,4 dimethylheptane (hydrogenated propylene trimer), 2,4,6-trimethyl nonane (hydrogenated propylene tetramer), 3 methylheptane (hydrogenated 1-butene dimer). In a sub-embodiment, the process used for making the at least one isoalkane involve metallocene catalyzed olefin oligomerisation.

In one embodiment, the process to make an alkylate base oil additionally comprises, after the dehydrating step (b), isolating a purified olefin from an unconverted alcohol and the at least one dimeric ketone and performing the alkylating with the purified olefin. Isolation of the purified olefin can be accomplished by exploiting that the olefin has higher hydrocarbon solubility and lower melting point than the alcohol and ketone it is made from. (see Example 8). Alternatively, the higher polarity of the alcohol and ketone may be exploited by passing the mixture of crude olefin containing unconverted alcohol and ketone optionally in a hydrocarbon solvent over a polar solid sorbent such as silica or alumina that preferentially adsorbs the polar alcohols and ketones while the olefins pass through and may be isolated in purified form from the effluent from the adsorption step.

The alkylating can be done using any suitable alkylation catalyst. In one embodiment, the alkylation catalyst is selected from the group consisting of an acidic ionic liquid, a sulfuric acid, a hydrofluoric acid, a trifluoromethanesulfonic acid, and a zeolite.

Acidic Ionic Liquid

Examples of acidic ionic liquid catalysts and their use for alkylation of paraffins with olefins are taught, for example, in U.S. Pat. Nos. 7,432,408 and 7,432,409, 7,285,698, and U.S. patent application Ser. No. 12/184,069, filed Jul. 31, 2008. In one embodiment, the acidic ionic liquid is a composite ionic liquid catalyst, wherein the cations come from a hydrohalide of an alkyl-containing amine or pyridine, and the anions are composite coordinate anions coming from two or more metal compounds.

The most common acidic ionic liquids are those prepared from organic-based cations and inorganic or organic anions. The acidic ionic liquid is composed of at least two components which form a complex. The acidic ionic liquid comprises a first component and a second component. The first component of the acidic ionic liquid will typically comprise a Lewis acid compound selected from components such as Lewis acid compounds of Group 13 metals, including aluminum halides, alkyl aluminum dihalides, gallium halide, and alkyl gallium halide (see the Periodic Table, which defines the elements that are Group 13 metals). Other Lewis acid compounds besides those of Group 13 metals may also be used. In one embodiment the first component is aluminum halide or alkyl aluminum dihalide. For example, aluminum trichloride (AlCl₃) may be used as the first component for preparing the ionic liquid catalyst. In one embodiment, the alkyl aluminum dihalides that can be used can have the general formula Al₂X₄R₂, where each X represents a halogen, selected for example from chlorine and bromine, each R represents a hydrocarbyl group comprising 1 to 12 atoms of carbon, aromatic or aliphatic, with a branched or a linear chain. Examples of alkyl aluminum dihalides include dichloromethylaluminum, dibromomethylaluminum, dichloroethylaluminum, dibromoethylaluminum, dichloro n-hexylaluminum, dichloroisobutylaluminum, either used separately or combined.

The second component making up the acidic ionic liquid can be an organic salt or mixture of salts. These salts may be characterized by the general formula Q+A−, wherein Q+ is an ammonium, phosphonium, boronium, oxonium, iodonium, or sulfonium cation and A− is a negatively charged ion such as Cl⁻, Br⁻, ClO₄⁻, NO₃⁻, BF₄⁻, BCl₄⁻, PF₆⁻, SbF₆⁻, AlCl₄⁻, Al₂Cl₇⁻, Al₃Cl₁₀⁻, GaCl₄⁻, Ga₂Cl₇⁻, Ga₃Cl₁₀⁻, AsF₆⁻, TaF₆⁻, CuCl₂⁻, FeCl₃⁻, AlBr₄⁻, Al₂Br₇⁻, Al₃Br₁₀⁻, SO₃CF₃⁻, and 3-sulfurtrioxyphenyl.

In one embodiment the second component is selected from those having quaternary ammonium halides containing one or more alkyl moieties having from about 1 to about 9 carbon atoms, such as, for example, trimethylammonium hydrochloride, methyltributylammonium, 1-butyl pyridinium, or alkyl substituted imidazolium halides, such as for example, 1-ethyl-3-methyl-imidazolium chloride.

In one embodiment, the acidic ionic liquid comprises a monovalent cation selected from the group consisting of a pyridinium ion, an imidazolium ion, a pyridazinium ion, a pyrazolium ion, an imidazolinium ion, a imidazolidinium ion, an ammonium ion, a phosphonium ion, and mixtures thereof. Examples of possible cations (Q+) include a butylethylimidazolium cation [beim], a butylmethylimidazolium cation [bmim], butyldimethylimidazolium cation [bmmim], decaethylimidazolium cation [dceim], a decamethylimidazolium cation [dcmim], a diethylimidazolium cation [eeim], dimethylimidazolium cation [mmim], an ethyl-2,4-dimethylimidazolium cation [e-2,4-mmim], an ethyldimethylimidazolium cation [emmim], an ethylimidazolium cation [eim], an ethylmethylimidazolium [emim] cation, an ethylpropylimidazolium cation [epim], an ethoxyethylmethylimidazolium cation [etO-emim], an ethoxydimethylimidazolium cation [etO-minim], a hexadecylmethylimidazolium cation [hexadmim], a heptylmethylimidazolium cation [hpmim], a hexaethylimidazolium cation [hxeim], a hexamethylimidazolium cation [hxmim], a hexadimethylimidazolium cation [hxmmim], a methoxyethylmethylimidazolium cation [meO-emim], a methoxypropylmethylimidazolium cation [meO-prmim], a methylimidazolium cation [mim], dimethylimidazolium cation [mmim], a methylnonylimidazolium cation [mnim], a methylpropylimidazolium cation [mpim], an octadecylmethylimidazolium cation [octadmim], a hydroxylethylmethylimidazolium cation [OH-emim], a hydroxyloctylmethylimidazolium cation [OH-omim], a hydroxylpropylmethylimidazolium cation [OH-prmim], an octylmethylimidazolium cation [omim], an octyldimethylimidazolium cation [ommim], a phenylethylmethylimidazolium cation [ph-emim], a phenylmethylimidazolium cation [ph-mim], a phenyldimethylimidazolium cation [ph-mmim], a pentylmethylimidazolium cation [pnmim], a propylmethylimidazolium cation [prmim], a 1-butyl-2-methylpyridinium cation[1-b-2-mpy], 1-butyl-3-methylpyridinium cation[1-b-3-mpy], a butylmethylpyridinium [bmpy] cation, a 1-butyl-4-dimethylacetylpyridinium cation [1-b-4-DMApy], a 1-butyl-4-35 methylpyridinium cation[1-b-4-mpy], a 1-ethyl-2-methylpyridinium cation[1-e-2-mpy], a 1-ethyl-3-methylpyridinium cation[1-e-3-mpy], a 1-ethyl-4-dimethylacetylpyridinium cation[1-e-4-DMApy], a 1-ethyl-4-methylpyridinium cation[1-e-4-mpy], a 1-hexyl-5 4dimethylacetylpyridinium cation[1-hx-4-DMApy], a 1-hexyl-4-methylpyridinium cation[1-hx-4-mpy], a 1-octyl-3-methylpyridinium cation[1-o-3-mpy], a 1-octyl-4-methylpyridinium cation[1-o-4-mp y], a 1-propyl-3-methylpyridinium cation[1-pr-3-mpy], a 1-propyl-4-methylpyridinium cation[1-pr-4-mpy], a butylpyridinium cation [bpy], an ethylpyridinium cation [epy], a heptylpyridinium cation [hppy], a hexylpyridinium cation [hxpy], a hydroxypropylpyridinium cation [OH-prpy], an octylpyridinium cation [opy], a pentylpyridinium cation [pnpy], a propylpyridinium cation [prpy], a butylmethylpyrrolidinium cation [bmpyr], a butylpyrrolidinium cation [bpyr], a hexylmethylpyrrolidinium cation [hxmpyr], a hexylpyrrolidinium cation [hxpyr], an octylmethylpyrrolidinium cation [ompyr], an octylpyrrolidinium cation [opyr], a propylmethylpyrrolidinium cation [prmpyr], a butylammonium cation [b-N], a tributylammonium cation [bbb-N], a tetrabutylammonium cation [bbbb-N], a butylethyldimethylammonium cation [bemm-N], a butyltrimethylammonium cation [bmmm-N], a N,N,N-trimethylethanolammonium cation [choline], an ethylammonium cation [e-N], a diethylammonium cation [ee-N], a tetraethylammonium cation [eeee-N], a tetraheptylammonium cation [hphphphp-N], a tetrahexylammonium cation [hxhxhxhx-N], a methylammonium cation [m-N], a dimethylammonium cation [mm-N], a tetramethylammonium cation [mmmm-N], an ammonium cation [N], a butyldimethylethanolammonium cation [OHe-bmm-N], a dimethylethanolammonium cation [OHe-mm-N], an ethanolammonium cation [OHe—N], an ethyldimethylethanolammonium cation [OHe-emm-N], a tetrapentylammonium cation [pnpnpnpn-N], a tetrapropylammonium cation [prprprpr-N], a tetrabutylphosphonium cation [bbbb-P], a tributyloctylphosphonium cation [bbbo-P], or combinations thereof.

In one embodiment, the second component is selected from those having quaternary phosphonium halides containing one or more alkyl moieties having from 1 to 12 carbon atoms, such as, for example, trialkyphosphonium hydrochloride, tetraalkylphosphonium chlorides, and methyltrialkyphosphonium halide.

In one embodiment, the acidic ionic liquid comprises an unsubstituted or partly alkylated ammonium ion.

In one embodiment, the acidic ionic liquid is chloroaluminate or a bromoaluminate. In one embodiment the acidic ionic liquid is a quaternary ammonium chloroaluminate ionic liquid having the general formula RR′R″NH+Al₂Cl₇, wherein R, R′, and R″ are alkyl groups containing 1 to 12 carbons. Examples of quaternary ammonium chloroaluminate ionic liquids are an N-alkyl-pyridinium chloroaluminate, an N-alkyl-alkylpyridinium chloroaluminate, a pyridinium hydrogen chloroaluminate, an alkyl pyridinium hydrogen chloroaluminate, a di alkyl-imidazolium chloroaluminate, a tetra-alkyl-ammonium chloroaluminate, a tri-alkyl-ammonium hydrogen chloroaluminate, or a mixture thereof.

The presence of the first component should give the acidic ionic liquid a Lewis or Franklin acidic character. Generally, the greater the mole ratio of the first component to the second component, the greater is the acidity of the acidic ionic liquid.

For example, a typical reaction mixture to prepare n-butyl pyridinium chloroaluminate ionic liquid is shown below:

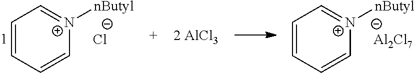

In one embodiment, the acidic ionic liquid utilizes a co-catalyst to provide enhanced or improved alkylation activity. Examples of co-catalysts include alkyl halide or hydrogen halide. A co-catalyst can comprise, for example, anhydrous HCl or organic chloride (see, e.g., U.S. Pat. No. 7,495,144 to Elomari, and U.S. Pat. No. 7,531,707 to Harris et al.). When organic chloride is used as the co-catalyst with the acidic ionic liquid, HCl may be formed in situ in the apparatus either during the alkylating or during post-processing of the output of the alkylating. In one embodiment, the alkylating with the acidic ionic liquid is conducted in the presence of a hydrogen halide, e.g., HCl.

The alkyl halides that may be used include alkyl bromides, alkyl chlorides and alkyl iodides. Such alkyl halides include but are not limited to iospentyl halides, isobutyl halides, t-butyl halides, n-butyl halides, propyl halides, and ethyl halides. Alkyl chloride versions of these alkyl halides can be preferable when chloroaluminate ionic liquids are used. Other alkyl chlorides or alkyl halides having from 1 to 8 carbon atoms can be also used. The alkyl halides may be used alone or in combination.

When used, the alkyl halide or hydrogen halide co-catalysts are used in catalytic amounts. In one embodiment, the amounts of the alkyl halides or hydrogen halide should be kept at low concentrations and not exceed the molar concentration of the AlCl₃ in the acidic ionic liquid. For example, the amounts of the alkyl halides or hydrogen halide used may range from 0.05 mol %-100 mol % of the Lewis acid AlCl₃ in the acidic ionic liquid in order to keep the acidity of the acidic ionic liquid catalyst at the desired performing capacity.

Zeolites for Alkylating

Zeolites useful for alkylating isoalkanes include large pore zeolites such as for instance zeolite X and zeolite Y and zeolite beta, in their proton form or rare earth exchanged form.

### EXAMPLES

### Example 1: Ketonization of Lauric Acid (Dodecanoic Acid, Fatty Acid) to 12-Tricosanone (Laurone, Ketone) Using an Alumina Catalyst

The ketonization of lauric acid (dodecanoic acid) to 12-tricosanone (laurone, ketone) was catalyzed by an alumina catalyst operated in a fixed bed continuously fed reactor at ambient pressure, at a temperature range of 770 to 840° C., and with a feed rate that gave a liquid hourly space velocity (LHSV) of 0.62 hr⁻¹ to 0.64 h⁻¹. The conversion of lauric acid to laurone was calculated based on the composition of the product, as determined by gas chromatography (GC) using a flame ionization detector (FID).

The freshly loaded new alumina catalyst was calcined in the reactor at 482° C. (900° F.) with a stream of dry nitrogen (2 volumes of nitrogen per volume of catalyst per minute) for 2 hours. Then the temperature was lowered to 410° C. (770° F.), the nitrogen stream was stopped, and the lauric acid feed was introduced into the reactor. Product composition analysis showed that the fresh catalyst operating at 410° C., LHSV=0.62 to 0.64 h⁻¹, gave a lauric acid conversion of 62 to 66 wt %.

The reactor effluent was split in a continuously operated stripping column from which the laurone product was isolated as a bottom cut containing less than 1 wt % unconverted lauric acid. The unconverted fatty acid (lauric acid) taken overhead from the stripping column was recycled to the reactor, except for a small amount (<5 wt % relative to the fresh fatty acid feed stock) of light cracked products. The light cracked products were predominantly n-alkanes and linear alpha olefins. The light cracked products were withdrawn from the stripping column as the only by-product stream.

### Example 2: Hydrogenation of 12-Tricosanone (Laurone, Ketone) to 12-Tricosanol (Corresponding Alcohol) Over Ruthenium/Carbon Catalyst

12-tricosanone (laurone, ketone) prepared as described in Example 1 was hydrogenated over a carbon supported ruthenium catalyst to make the corresponding alcohol, 12-tricosanol as described here.

800 g of the 12-tricosanone (laurone, ketone) was loaded into a 1 liter stirred batch autoclave together with 1 g of a catalyst having 5 wt % ruthenium on carbon. The mixture of the 12-tricosanone and catalyst was put under 1500 psig (10342 kPa) hydrogen pressure, stirred, and heated to 200° C. Hydrogen was added as it was consumed in order to maintain the hydrogen pressure in the reactor during the run. After 23 hours the reaction was stopped and the reactor contents withdrawn and filtered to yield the 12-tricosanol product. Proton nuclear magnetic resonance (NMR) indicated that the conversion was about 89 wt % and the selectivity to the alcohol was greater than 90 wt %, with the corresponding alkane, tricosane, being the only by-product.

### Example 3: Hydrogenation of 12-Tricosanone (Laurone, Ketone) to 12-Tricosanol (Corresponding Alcohol) Over Ruthenium/Tin/Carbon Catalyst

2185 g of 12-tricosanone prepared as described in Example 1 was loaded into a 1 gallon stirred autoclave with 3 g of a catalyst comprising 5 wt % ruthenium on a tin promoted carbon support. The mixture of the 12-tricosanone and catalyst was put under 1500 psig (10342 kPa) hydrogen pressure, stirred, and heated to 200° C. Hydrogen was added as it was consumed in order to maintain the hydrogen pressure in the reactor during the run. After 36 hours the reaction was stopped and the reactor contents withdrawn and filtered to yield the 12-tricosanol product. Proton nuclear magnetic resonance (NMR) indicated that the conversion was about 93 wt % and the selectivity to 12-tricosanol was about 95 wt %. Later analysis of the olefin isolated by dehydration of the 12-tricosanol product (see Example 7) showed that the product contained less than 2 wt % alkane, indicating greater than 98 wt % selectivity in this hydrogenation step.

### Example 4: Hydrogenation of 12-Tricosanone (Laurone, Ketone) to 12-Tricosanol (Corresponding Alcohol) Over Pt/Carbon Hydrogenation Catalyst

12-tricosanone (laurone, ketone) prepared as described in Example 1 was hydrogenated over a carbon supported platinum catalyst to make the corresponding alcohol, 12-tricosanol as described here.

The 12-tricosanone was introduced as a liquid flow (4.1-4.4 g/hr, 12-13 mmoles/hr) together with hydrogen (100 Nml/min, 250 mmoles/hr) to a fixed reactor holding 7 ml of a catalyst comprising 0.5 wt % platinum on carbon. The amount of the catalyst was 3.5 g, and the catalyst had a particle size of 0.3 to 1 mm. The pressure was held at 1500 psig (10342 kPa). The liquid products were collected after the reaction and analyzed by GC. The liquid product stream contained three components: 1) unconverted 12-tricosanone, 2) 12-tricosanol, and 3) the corresponding n-alkane, n-tricosane. The n-tricosane was present only in trace amounts.

At a reaction temperature from 450 to 470° F., the GC analysis of the product showed a conversion of 12-tricosanone of 80 to 87 wt %, and a selectivity to 12-tricosanol of 98.9 to 99.4 wt %. The remaining 0.6 to 1.1 wt % of the product was n-tricosane formed by hydro-deoxygenation of the alcohol.

### Example 5: Hydrogenation of Coconut Fatty Acid Derived Ketones to a Mixture of Linear Secondary Alcohols

A sample of saturated fatty acids from coconut oil contained a mixture of C8-C14 fatty acids, with C12 and C14 fatty acids being the predominant components. The saturated fatty acids from coconut oil were reacted under ketonization conditions over an alumina catalyst at a temperature of 790 to 820° F. and at atmospheric pressure to prepare a product mixture. A mixture of C19-C27 ketones with greater than 90 wt % ketones and less than 1 wt % unconverted fatty acids were isolated from the product mixture.

The above described coconut-derived ketone mixture was converted to the corresponding linear secondary alcohols by hydrogenation using a fixed bed of a catalyst comprising 0.5 wt % platinum on carbon. The hydrogenation was carried out at 1500 psig (10342 kPa) pressure and at a temperature from 450 to 460° F. The hydrogenation achieved about 90 wt % ketone conversion to a mixture of the corresponding linear secondary alcohols (80 to 90 wt % selectivity, and the corresponding alkanes (10 to 20 wt % selectivity) based on GC analysis of the mixed products from the hydrogenation. Improved selectivity for the linear secondary alcohol products can be achieved through optimization of the hydrogenation reactor and flow distribution of the ketone mixture over the catalyst.

### Example 6: Hydrogenation of Beef Tallow Fatty Acid Derived Ketones to a Mixture of C29-C35 Linear Secondary Alcohols

A sample of a saturated fatty acid mixture was prepared from beef tallow and consisted predominantly of stearic acid (octadecanoic acid, about 45 wt %) and palmitic acid (hexadecanoic acid, about 45 wt %), and had smaller amounts of myristic acid (tetradecanoic acid, about 5 wt %) and other fatty acids. The sample of saturated fatty acid mixture prepared from beef tallow was TRT1655, from Twin Rivers Technologies, Quncy Mass. The sample of saturated fatty acid mixture prepared from beef tallow was processed over an alumina catalyst at a temperature from 800 to 810° F. and at atmospheric pressure to produce a reactor effluent from which a mixture of predominantly C29-C35 ketones, with less than 0.15 wt % fatty acids, was isolated.

The isolated mixture of predominantly C29-35 ketones was hydrogenated over a catalyst comprising 0.5 wt % platinum on carbon. The hydrogenation conditions included a temperature of 343.4° C. (650° F.), a hydrogen pressure of 1588 psig (10949 kPa), and a LHSV of 0.48 hr-1. The hydrogenation yielded the corresponding C29-C35 linear secondary alcohol mixture with a ketone conversion of about 82 wt % and a selectivity above 99 wt % C29-C35 linear secondary alcohols.

### Example 7: Dehydration of 12-Tricosanol to a Mixture of Predominantly Cis, Trans-11-Tricosene Over an Alumina Catalyst

The 12-tricosanol, made as described in Example 3, was used without further purification. The 12-tricosanol was fed at a LHSV of 0.4-0.53 hr⁻¹ to a fixed bed reactor operating at 343.4° C. (650° F.) and atmospheric pressure and containing 50 ml freshly regenerated alumina catalyst of the same kind used for the ketonization described in Example 1. The regeneration of the alumina catalyst was done by contacting the catalyst with an oxidizing gas to remove coke and further contacting the catalyst with steam, as described in a U.S. patent application Ser. No. 14/540,723, filed Nov. 13, 2014.

GC and NMR analysis of the product withdrawn from the fixed bed reactor, after ejection of water, showed a 12-tricosanol conversion of 87 to 90 wt %, and near quantitative (about 99 wt %) selectivity to a mixture of cis and trans 11-tricosene, with only traces of other olefin isomers. The GC and NMR analysis showed the presence of 2 wt % tricosane relative to the combined tricosane and tricosane, presumably carried over from the hydrogenation step described in Example 3.

### Example 8: Isolation of Tricosene from Crude Tricosene Product

Several efficient methods can be used for separation of tricosane from unconverted 12-tricosanol and 12-tricosanone. One method exploited the far higher solubility of the olefin in light alkane solvents at low temperature. It was possible to perform the separation of the tricosene by dissolving the mixture of tricosene, tricosanol, and tricosanone in hexane and cooling the dissolved mixture to −20° C. to precipitate out essentially all of the unconverted tricosanol and tricocanone. The solid precipitates were removed by filtration and after subsequent evaporation of the hexane solvent, a purified tricosene product containing 0.02 wt % tricosanol and 0.9 wt % tricosanone was isolated.

Another method used for separating the tricosane removed the tricosanol and tricosanone from the tricosane by passing a solution of the crude mixture in a hydrocarbon solution through a column of dry silica gel sorbent. The dry silica gel sorbent selectively adsorbed the tricosanol and tricosanone, and left the tricosane with essentially no tricosanol and only traces of tricosanone.

Although this example speaks of our experiments with tricosene, the separation methods described in this example can also be used to isolate other olefins prepared in similar manners from other fatty acid derived ketones.

### Example 9: Alkylation of Isopentane with Tricosene Using an Acidic Ionic Liquid Catalyst

50 ml n-butylpyridinium heptachlorodialuminate ionic liquid catalyst and 700 ml isopentane was placed in a 2 liter mechanically-stirred reaction flask under an inert atmosphere (nitrogen) and cooled on an ice water bath. While maintaining a reaction temperature in the range of 3-5° C., a mixture of about 94 g tricosene and 1 g t-butyl chloride in 191 g isopentane was slowly added to the reaction flask over a period of 50 minutes. The reaction mixture was stirred for another 15 minutes and then allowed to settle. The hydrocarbon phase was decanted off, treated with a small amount of sodium bicarbonate (NaHCO₃) and water to make a clear colorless solution. 96.3 g of oil was isolated from the clear colorless solution by evaporation on a rotary evaporator (RotoVap) at 8 torr and 91° C. The isolated oil had the following properties, as shown in Table 2.

TABLE 2

| | |
|---|---|
| Viscosity Index | 157 |
| Kinematic Viscosity at 100° C., mm²/s | 10.58 |
| Kinematic Viscosity at 40° C., mm²/s | 63.38 |
| Pour Point, ° C. | −2 |

### Example 10: Alkylation of Farnesane with Tricosene Using an Acidic Ionic Liquid Catalyst

Farnesane was prepared by hydrogenation of farnesene (mixture of isomers, acquired from Sigma Aldrich) over a fixed bed of 20.7 wt % nickel on alumina catalyst (Johnson Matthey HTC500) at 320° F. and about 1700 psig (11721 kPa) using an LHSV of about 0.6-0.8 hr⁻¹.

400 ml of the prepared farnesane was combined with 40 ml n-butylpyridinium heptachlorodialuminate ionic liquid catalyst in a mechanically-stirred 2 liter reaction flask under inert atmosphere (nitrogen) and cooled to 4° C. on an ice bath. A mixture of 50 ml (39.6 g) tricosene and 0.5 ml t-butyl chloride was added to the reaction flask over a period of 50 minutes, while the reaction temperature was maintained at 3-5° C. After an additional 10 minutes the stirring was stopped, the ionic liquid phase was allowed to settle out, and the hydrocarbon phase was decanted off. The hydrocarbon phase was stirred with ice and enough sodium bicarbonate (NaHCO₃) to neutralize the residual acidic ionic liquid catalyst. Subsequently, the excess farnesane was distilled out at up to 149° C. and 2 torr on a RotoVap at 8 torr and 91° C., to isolate a yellow viscous oil. The isolated yellow viscous oil had the following properties, as shown in Table 3.

TABLE 3

| | |
|---|---|
| Viscosity Index | 129 |
| Kinematic Viscosity at 100° C., mm²/s | 11.16 |
| Kinematic Viscosity at 40° C., mm²/s | 79.93 |
| Pour Point, ° C. | −25 |

### Example 11: Alkylation of C6-C12 Isoalkanes with Tricosene Using an Acidic Ionic Liquid Catalyst

A sample of C6-C12 isoalkanes was a naphtha cut collected from the product made by hydrogenating light propylene oligomers made by metallocene catalyzed oligomerization of propylene. The sample of C6-C12 isoalkanes comprised methyl pentane, dimethyl heptane, and trimethyl nonane. Gas chromatographic analysis of the sample of C6-C12 isoalkanes showed roughly the following composition: 38 wt % 2-methylpentane, 52 wt % dimethylheptane, 8 wt % trimethylnonane, and 2 wt % heavier isoalkanes.

600 ml (384 g) of the sample of C6-C12 isoalkanes were combined with 40 ml n-butylpyridinium heptachlorodialuminate ionic liquid catalyst in a mechanically-stirred 2 liter reaction flask under inert atmosphere (nitrogen) and cooled to 2° C. on an ice bath. A mixture of 60 ml (47.6 g) tricosene and 0.6 ml t-butyl chloride was added to the reaction flask over a period of about 1 hour, while the reaction temperature was maintained at about 2° C. The stirring was stopped, the ionic liquid phase was allowed to settle out, and the hydrocarbon phase was decanted off. The hydrocarbon phase was treated with water and enough sodium bicarbonate (NaHCO₃) to neutralize the residual acidic ionic liquid catalyst. Subsequently, the excess C6-C9 isoalkanes were distilled out and the hydrocarbon phase was concentrated on a RotoVap at 9 torr and 92° C., to isolate a yellowish alkylate base oil.

A TBP analysis of the isolated yellowish alkylate base oil revealed that the isolated oil still contained significant amounts of material with a boiling point less than 200° C. The isolated yellowish alkylate base oil was then heated to 125° C. in a stream of nitrogen for about 1 hour to remove residual light hydrocarbons and produce a final product. The final product was an alkylate base oil having the properties as shown in Table 4.

TABLE 4

| | |
|---|---|
| Viscosity Index | 182 |
| Kinematic Viscosity at 100° C., mm²/s | 5.047 |
| Kinematic Viscosity at 40° C., mm²/s | 20.94 |
| Pour Point, ° C. | −26 |

It is notable that the examples of alkylate base oil described herein were all done without any hydroisomerization. By eliminating the hydroisomerization, much less or no hydrocracking into lighter and less valuable products occurred.

The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

For the purposes of this specification and appended claims, unless otherwise indicated, all numbers expressing quantities, percentages or proportions, and other numerical values used in the specification and claims, are to be understood as being modified in all instances by the term “about.” Furthermore, all ranges disclosed herein are inclusive of the endpoints and are independently combinable. Whenever a numerical range with a lower limit and an upper limit are disclosed, any number falling within the range is also specifically disclosed. Unless otherwise specified, all percentages are in weight percent.

Any term, abbreviation or shorthand not defined is understood to have the ordinary meaning used by a person skilled in the art at the time the application is filed. The singular forms “a,” “an,” and “the,” include plural references unless expressly and unequivocally limited to one instance.

All of the publications, patents and patent applications cited in this application are herein incorporated by reference in their entirety to the same extent as if the disclosure of each individual publication, patent application or patent was specifically and individually indicated to be incorporated by reference in its entirety.

This written description uses examples to disclose the invention, including the best mode, and also to enable any person skilled in the art to make and use the invention. Many modifications of the exemplary embodiments of the invention disclosed above will readily occur to those skilled in the art. Accordingly, the invention is to be construed as including all structure and methods that fall within the scope of the appended claims. Unless otherwise specified, the recitation of a genus of elements, materials or other components, from which an individual component or mixture of components can be selected, is intended to include all possible sub-generic combinations of the listed components and mixtures thereof.

The invention illustratively disclosed herein suitably may be practiced in the absence of any element which is not specifically disclosed herein.

It is claimed:

1. A process to make an alkylate base oil having a viscosity index greater than or equal to 90, comprising:
a. converting an at least one dimeric ketone to an at least one alcohol;
b. dehydrating the at least one alcohol to make one or more corresponding olefins; and
c. alkylating at least one isoalkane with the one or more corresponding olefins to form the alkylate base oil.

2. The process of claim 1 wherein the alkylate base oil has the viscosity index greater than or equal to 120.

3. The process of claim 1, wherein the alkylate base oil is an API Group III base oil.

4. The process of claim 1, wherein the alkylate base oil has a kinematic viscosity at 100° C. from 3.0 to 20 mm²/s.

5. The process of claim 1, wherein the alkylate base oil has a higher molecular weight, a lower pour point, and a lower cloud point than the at least one dimeric ketone.

6. The process of claim 1, wherein the alkylate base oil has a bromine index less than 1000 and a kinematic viscosity at 100° C. greater than 3 mm²/s.

7. The process of claim 1, wherein the alkylating introduces branching into the alkylate base oil at a central position such that the alkylate base oil has a pour point less than −15° C.

8. The process of claim 1, wherein the at least one dimeric ketone is derived from a biological source.

9. Process according to claim 1, wherein the at least one dimeric ketone is prepared by ketonization of one or more carboxylic acids.

10. The process of claim 1, wherein the converting is done by contacting the at least one dimeric ketone with a hydrogen and a solid hydrogenation catalyst.

11. The process of claim 10, wherein the solid hydrogenation catalyst is a carbon supported metal hydrogenation catalyst.

12. The process of claim 11, wherein the carbon supported metal hydrogenation catalyst comprises from 0.1 to 10 wt % hydrogenation metal, and a conversion of the at least one dimeric ketone to the at least one alcohol is greater than 80 wt %.

13. The process of claim 12, wherein the carbon supported metal hydrogenation catalyst comprises one or more metals from the group consisting of Ru, Pt, and Cu.

14. The process of claim 1, wherein the converting has a selectivity to make an at least one corresponding alcohol of 80 wt % or more.

15. The process of claim 1, wherein the dehydrating is done with a dehydration catalyst comprising at least 90 wt % alumina.

16. The process of claim 1, wherein the one or more corresponding olefins have a carbon number from C11 to C43.

17. The process of claim 16, wherein the one or more corresponding olefins have the carbon number from C19 to C35.

18. The process of claim 1, additionally comprising hydroisomerizing the alkylate base oil with a hydroisomerization catalyst to lower a pour point of the alkylate base oil.

19. The process of claim 1, wherein no hydroisomerization is used.

20. The process of claim 1, wherein the at least one isoalkane has from four to 36 carbon atoms.

21. The process of claim 1, wherein the at least one isoalkane comprises a farnesane or an isopentane.

22. The process of claim 1, wherein the at least one isoalkane comprises a mixture of naphtha range hydrocarbons.

23. The process of claim 1, wherein the alkylating is done using an alkylation catalyst selected from the group consisting of an acidic ionic liquid, a sulfuric acid, a hydrofluoric acid, a trifluoromethanesulfonic acid, and a zeolite.

24. The process of claim 23, wherein the alkylation catalyst is the acidic ionic liquid.

25. The process of claim 24, wherein the process additionally comprises passing the one or more corresponding olefins over an olefin isomerization catalyst to shift a double bond position to another internal position, without structurally introducing branching in the one or more corresponding olefins prior to the alkylating.

26. The process of claim 1, additionally comprising, after step b): isolating a purified olefin from an unconverted alcohol and the at least one dimeric ketone and performing the alkylating with the purified olefin.

27. The process of claim 1, additionally comprising blending the alkylate base oil with at least one additive to make a finished lubricant.
